(19) United States
(12) Patent Application Publication
(10) Pub. No.: US 2022/0347012 A1
(43) Pub. Date: Nov. 3, 2022

(54) DEVICE FOR THE REFRACTIVE SURGERY OF AN EYE AND A PROCESS FOR MONITORING THE FUNCTIONALITY THEREOF

(71) Applicant: Carl Zeiss Meditec AG, Jena (DE)

(72) Inventors: Andreas Wirth, Simbach am Inn (DE); Gregor Stobrawa, Jena (DE)

(21) Appl. No.: 17/634,714

(22) PCT Filed: Aug. 11, 2020
(86) PCT No.: PCT/EP2020/072551

§ 371 Date: Feb. 11, 2022

(30) Foreign Application Priority Data
Aug. 13, 2019 (DE) 10 2019 121 731.8

(51) Int. Cl. A61F 9/008

(52) U.S. Cl. CPC A61F 9/008; A61F 9/00804; A61F 9/00827; A61F 2009/00872

(57) ABSTRACT

The present invention relates to a method for monitoring a functionality of an apparatus (100) for refractive surgery on an eye (2) using a laser beam (4). The method comprises a determination of at least one parameter of a preparation and/or implementation of a method for refractive surgery, wherein the preparation and/or the implementation of the method are carried out at least in part by the apparatus for refractive surgery using the laser beam (4). Further, the method comprises an analysis of the functionality of the apparatus (100) on the basis of the determined parameter of the preparation and/or the implementation of the method for refractive surgery. In this case, the at least one parameter characterizes one or more of the following properties of the preparation and/or implementation of the method for refractive surgery:
a provision of the laser beam (4) by the apparatus (100);
an effect on the eye (2) by the apparatus (100);
an external influence on the preparation and/or the implementation of the method for refractive surgery from outside of the apparatus (100); and
a suitability of the effect on the eye (2) by the laser beam (4) for the further method of the refractive surgery on the eye (2).

Moreover, the invention relates to a control unit (14) and an apparatus (100) for refractive surgery on an eye (2).

The present invention relates to a method for monitoring a functionality of an apparatus for refractive surgery on an eye, in particular using a laser beam. The invention further relates to a control unit and an apparatus for refractive surgery on an eye. In particular, the invention is found in the field of refractive surgery and particularly preferably in the field of lenticule extraction by way of a small incision for the purposes of correcting a refractive error.

The prior art has disclosed methods which by means of a minimally invasive laser correction relate to the refractive surgical correction of refractive errors. In the process, a lenticule is detached within the cornea of the eye and subsequently removed by surgery through a small incision with a length of approximately 2 mm to 4 mm. Such a method was developed by the applicant and is known as Small Incision Lenticule Extraction, for example. Suitable apparatuses going by the trade name of VisuMax are commercially available from the applicant.

To correct the refractive error of the eye in optimal fashion, the greatest possible precision is required when detaching the lenticule in the cornea by means of the laser beam. This requires the apparatus for refractive surgery on the eye to be correctly configured and adjusted in order to minimize or even entirely eliminate possible deviations between the desired correction and the refractive correction actually brought about.

It is therefore desirable to provide continuous monitoring of the functionality of the apparatus for refractive surgery.

Procedures for monitoring a functionality of a machine, which make use of predictive maintenance in order thus to monitor the state or the functionality of the machine and already recognize possible maintenance needs in advance, are generally known from the prior art. This follows the object of reducing downtimes of the machine on account of a faulty function or damage, recognizing maintenance needs already in advance and performing the maintenance before there is damage or loss of functionality. Typically, one or more measurement values directly accessible on or in the machine are measured and used in this case to determine the need for maintenance and thus prevent an outage of the machine and keep the machine in a state that is as process-compliant as possible. The measurement values are typically determined using the properties accessible in the system, for instance a temperature and/or a voltage and/or a required power consumption by the machine, for instance by a motor of the machine. Subsequently, these measurement values can be correlated with possibly occurring problems of the functionality, for instance an operational outage. By way of example, increased wear and/or an imminent operational outage can be deduced from a voltage or power requirement that increases over time in the case of otherwise unchanged operation, and the necessity of maintenance for remedying the flaw can be derived.

However, such a method for predictive maintenance known from the prior art is restricted to those machines which provide suitable machine-internal and machine-measurable parameters which permit a correlation with problems. Other apparatuses, which do not provide suitable, machine-internal and machine-measurable parameters, or which do not provide a sufficient amount of such parameters, can therefore not be monitored using a conventional method for predictive maintenance.

It is the object of the invention to provide a method for monitoring a functionality of an apparatus for refractive surgery, which reliably recognizes problems with the functionality and which prevents a faulty operation and/or operational outages.

This object is achieved by a method for monitoring a functionality of an apparatus for refractive surgery, a control unit and an apparatus which have the features of the respective independent claims. Preferred embodiments are the subject matter of the dependent claims and the following description.

In a first aspect, the invention relates to a method for monitoring a functionality of an apparatus for refractive surgery on an eye using a laser beam. The method comprises a determination of at least one parameter of a preparation and/or implementation of a method for refractive surgery, wherein the preparation and/or the implementation of the method are carried out at least in part by the apparatus for refractive surgery using the laser beam. Further, the method comprises an analysis of the functionality of the apparatus on the basis of the ascertained parameter of the preparation and/or the implementation of the method for refractive surgery. In this case, the at least one parameter characterizes one or more of the following properties of the preparation and/or implementation of the method for refractive surgery:
a provision of the laser beam by the apparatus;
an effect on the eye by the apparatus;
an external influence on the preparation and/or the implementation of the method for refractive surgery from outside of the apparatus; and
a suitability of the effect on the eye by the laser beam for the further method of the refractive surgery on the eye.

The method can preferably be implemented (in automated fashion) by an appropriately configured control unit.

In a further aspect, the invention relates to a control unit which is configured to cause an apparatus for refractive surgery on an eye using a laser beam to implement the steps mentioned below. In a first step there is a determination of at least one parameter of a preparation and/or implementation of a method for refractive surgery, wherein the preparation and/or the implementation of the method are carried out at least in part by the apparatus for refractive surgery using the laser beam. In a second step there is an analysis of the functionality of the apparatus on the basis of the determined parameter of the preparation and/or the implementation of the method for refractive surgery. In this case, the at least one parameter characterizes one or more of the following properties of the preparation and/or implementation of the method for refractive surgery:
a provision of the laser beam by the apparatus;
an effect on the eye by the apparatus;
an external influence on the preparation and/or the implementation of the method for refractive surgery from outside of the apparatus; and
a suitability of the effect on the eye by the laser beam for the further method of the refractive surgery on the eye.

In a further aspect, the invention relates to an apparatus for refractive surgery on an eye, wherein the apparatus comprises a control unit according to the invention.

Monitoring the functionality of an apparatus can relate in particular to the monitoring of the functionality during the operation of the apparatus, that is to say while the apparatus is used for refractive surgery on the eye and/or for the preparation of the same. Expressed differently, monitoring the functionality may comprise the monitoring during a single or individual refractive surgical application in order to monitor and/or ensure the correct operation and the correct implementation of the refractive surgical intervention by the apparatus. Alternatively or in addition, the monitoring of the functionality may comprise monitoring over a multiplicity of refractive surgical interventions or applications and, in particular, comprise collecting and/or determining statistical data relating to the parameters and/or the functionality of the apparatus. Expressed differently, monitoring the functionality may be carried out in the style of long-term monitoring in order to be able to recognize and/or analyze a change in the parameters which arise over a multiplicity of refractive surgical interventions. Particularly preferably, monitoring the function may comprise both monitoring the function for one, more and/or all single or individual refractive surgical interventions and long-term monitoring over a multiplicity of refractive surgical interventions.

An apparatus for refractive surgery is preferably designed as an apparatus for correcting myopia and/or astigmatism of an eye. In particular, the apparatus comprises a laser source and/or receives a laser beam from an external, separate laser source. The laser source preferably comprises an excimer laser and/or a femtosecond laser, or is designed as such. The laser beam can preferably be provided as a continuous wave (cw) laser beam, or as a pulsed laser beam. Particularly preferably, the laser beam is provided as a pulse sequence of femtosecond laser pulses, by which the lenticule is detached from the surrounding cornea by exploiting cavitation. The apparatus is preferably configured to impinge the eye to be treated with the laser beam and preferably to detach a lenticule from the cornea of the eye. The refractive surgical method can preferably further comprise the surgical removal, particularly preferably the minimally invasive surgical removal, of the detached lenticule from the cornea. The surgical removal can preferably be carried out by surgeon independently of the apparatus, that is to say without support by the apparatus. Alternatively, the apparatus can preferably also be designed to assist the surgical removal of the detached lenticule.

In this case, a parameter of the preparation and/or implementation of the method for refractive surgery preferably is an objectively and/or subjectively determinable measurement variable, which can be measured before and/or during and/or after the preparation and/or implementation of the method and which is directly or indirectly suitable for the analysis of the functionality of the apparatus.

One or more or even all parameters can be determined by the control unit in the process. By way of example, it is possible to provide one or more sensors which are connected to the control unit and by which the control unit can determine the one or more parameters. Also, the control unit can be configured to additionally determine other measurement values. Here, the control unit can preferably comprise a processor and/or another type of computing unit in order to be able to implement the method, preferably in automated fashion. Also, the control unit can be configured to output, for example by way of a display, a request to a user to provide a value and/or parameter, for example by way of an entry with a keyboard and/or by way of a selection on the screen. The latter can lend itself for those parameters, in particular, which involve a subjective perception of the user and which cannot be objectively determined, or can only be objectively determined with difficulties, by means of sensors.

Implementing a method for refractive surgery in particular comprises the impingement of the eye with the laser beam by the apparatus. Expressed differently, the implementation of the method preferably comprises the application of the laser beam to the eye. The implementation of the method therefore preferably comprises the detachment of the lenticule from the surrounding cornea of the eye. A possible subsequent surgical removal of the lenticule from the eye is not necessarily comprised by the implementation of the method for refractive surgery using the apparatus, but can by all means count as part of implementation of the method. Moreover, the implementation may comprise even further optional method steps, for instance rinsing or cleaning the eye or the surface of the cornea, which can optionally be implemented using the device.

The preparation of the method for refractive surgery preferably comprises an arrangement or docking of the apparatus on the eye to be treated, which facilitates the impingement of the eye with the laser beam with the necessary precision. Preferably, the apparatus can comprise a contact glass to this end, by means of which the apparatus can dock on the eye. The laser beam can preferably be subsequently applied to the eye through the contact glass. The preparation of the method can optionally include even further steps, for instance a suitable positioning and/or fixation of the patient and/or of the eye relative to the apparatus, and/or a preparation of the eye for the impingement with the laser beam. Further, the preparation can preferably comprise a calibration and/or an adjustment and/or an initialization and/or a (self) test of the apparatus, within the scope of which, for example, the positionability of the apparatus and/or of the laser beam and/or a power and/or intensity and/or fluence or energy flux of the laser beam is set and/or monitored.

The fact that the preparation and/or the implementation are carried out at least in part by the apparatus for refractive surgery using the laser beam means that the preparation and/or implementation may optionally comprise even further steps that are carried out without the use of the apparatus.

The invention offers the advantage of being able to increase the reliability of apparatuses for refractive surgery on an eye. In particular, this is achieved by virtue of, according to the invention, a method being provided for monitoring the functionality of apparatuses for refractive surgery, the method allowing a reliable analysis of the functionality on the basis of parameters.

In particular, the invention offers the advantage of being able to monitor and ensure the functionality of the apparatus in the style of predictive maintenance, even if there might not be a sufficient number of objective, system-internal and/or machine-measurable data or measurement values, for instance as in the case of conventional machines accessible to predictive maintenance. However, the invention offers the advantage of preferably being able to analyze the functionality of the apparatus even on the basis of those parameters that do not exclusively relate to objective and apparatus-internal measurement values. Rather, the invention offers the advantage that it is preferably also possible to use those parameters that indirectly facilitate deductions about the functionality of the apparatus and a corresponding analysis.

Preferably, the analysis of the functionality of the apparatus on the basis of the determined parameter comprises a comparison of the determined parameter of the preparation and/or the implementation of the method for refractive surgery on the basis of the determined parameter with a predetermined intended value and an assessment of the functionality of the apparatus for refractive surgery on the basis of the comparison of the determined parameter with the predetermined intended value. By way of example, the analysis of the functionality can be based on a deviation of the actually determined parameter from the specified intended value. This offers the advantage that deviations from a specified intended state can be recognized particularly reliably and the functionality can be monitored particularly accurately. By way of example, the analysis can be carried out in automated fashion by the control unit.

Preferably, the method further comprises an implementation of a measure for improving the functionality of the apparatus. By way of example, such a measure can consist of correcting and/or changing one or more settings of the apparatus in a suitable and preferably automated manner in order to improve the functionality. By way of example, such a measure can consist of the power of the laser beam being increased or reduced in an automated manner in order to improve or re-establish the functionality of the apparatus. This, too, can be undertaken preferably independently by the control unit. By way of example, the control unit can transmit one or more appropriate commands to the apparatus to this end. Alternatively, the control unit can output a notification, for example, to the user so that the latter can introduce necessary measures.

Alternatively or in addition, the method preferably further comprises a provision of a notification about the functionality of the apparatus. The notification can preferably be made available to a user of the apparatus and/or a maintenance device and/or the manufacturer. By way of example, such a notification can be output on a screen such that the user is informed during the use of the apparatus. Alternatively or in addition, for example a notification can be transferred via a network, for instance via the Internet and/or a mobile radio network, in order to inform a maintenance device situated at a distance from apparatus and/or the manufacturer of the notification. Preferably, the notification can contain information relating to the functionality of the apparatus. Preferably, it is possible to gather from the notification that there is or is not a need for servicing, or that this may be expected at a certain or indeterminate time in the future. This offers the advantage that, for example, the need for servicing can be determined without the presence of specifically schooled maintenance staff in situ and preferably without a downtime of the apparatus.

Alternatively or in addition, the method preferably comprises an adjustment or modification of an operation of the apparatus. This may comprise for example blocking the laser beam and/or deactivating the laser source and/or deactivating the entire apparatus. Preferably, such measures are only adopted if a particularly severe impairment of the functionality is recognized, within the scope of which for example endangerment of the patient and/or significant damage to the apparatus and/or other objects cannot be precluded and/or which requires quick or even immediate action. An exception in this case is the prevention of the “normal” start of the apparatus at the beginning of a corresponding method: There need not be a particularly severe impairment to this end: A significant deviation from the intended value prior to the “normal” start of the method preferably blocks the start of one of the following methods for refractive surgery.

Preferably, the determination of a parameter characterizing the provision of the laser beam by the apparatus comprises a determination of a state of a contact glass of the apparatus, wherein the laser beam is applicable to the eye through the contact glass. By way of example, the state of the contact glass can be determined to the effect of whether contamination of and/or damage to the contact glass is present, the contamination and/or damage influencing the functionality of the contact glass and accordingly influencing the functionality of the apparatus. In this case, the determination of the state can be carried out in automated fashion by the apparatus itself and/or by a user of the apparatus, for example following a corresponding notification and/or corresponding instructions, which are preferably provided by the apparatus. This offers the advantage of being able to reliably remedy an impairment due to a faulty contact glass.

Preferably, the determination of the state of the contact glass comprises a determination of optical structures in an image of optical radiation passing through the contact glass of the apparatus. This offers the advantage that the determination of the state of the contact glass can be carried out particularly reliably in an automated fashion in this way. By way of example, to this end, recording and evaluating an optical image of light passing through the contact glass can be used to determine the optical structures in the captured image. Particularly preferably, this can be carried out using hardware present in the apparatus in any case without additional hardware having to be provided therefor.

Preferably, the determination of the optical structures comprises a determination of optical edges in the image, wherein the determination of the optical edges preferably comprises a determination of edges running in a straight line and/or edges running in a circle. By way of example, this can be achieved by the application of optical filters. To this end, high or low spatial frequencies in the beam path of the image can be isolated or filtered, and only the remaining spatial frequencies can be examined for the presence of edges. Alternatively or in addition, an electronic image evaluation, for example using corresponding digital filters, can be used to determine the presence of edges. By way of example, the presence of edges running in straight lines may be an indication of hair and/or eyelashes adhering to the contact glass. The presence of edges running in a circle may be an indication for residue of fat and/or droplets and/or tears on the contact glass. Consequently, the presence of edges can be an indication that the contact glass should be cleaned and/or replaced, and/or that a notification to this effect should be provided and/or that the operation of the apparatus should be interrupted in severe cases. Additionally, the determined parameters can preferably be provided to the manufacturer of the apparatus and/or of the contact glass, for instance in order to cause or prompt a check of the manufactured contact glasses and/or individual batches.

Preferably, the determination of a parameter characterizing the effect on the eye by the apparatus comprises a determination of a docking time of the apparatus, in particular the contact glass, on the eye and/or a determination of a number of necessary docking attempts until the apparatus successfully docks on the eye. By way of example, an increased number of required docking attempts may be an indication for a restless patient and/or for a technical problem of the apparatus, for instance a faulty and/or contaminated and/or damaged contact glass. Thus, for example, an analysis may come to the conclusion that a notification for the user to exchange and/or clean the contact glass may be advantageous.

Preferably, the determination of a parameter characterizing the effect on the eye by the apparatus comprises a determination of arising optical structures in an image of the eye while the laser beam has an effect on the eye, wherein the arising optical structures preferably comprise bright spots and/or dark spots. By way of example, the arising optical structures may be indications for the occurrence and/or cessation of an interaction of the laser beam with the eye. Thus, preferably, the occurrence of bright spots can preferably be a result of a scattering center at the location of interaction between the laser beam and the eye, which may represent an indication for cavitation or bubble formation. Although cavitation may be desired for the effect on the eye by the laser beam, the occurrence of bright spots may nevertheless be an indication for too much cavitation under certain circumstances. By way of example, the power of the laser beam can be reduced accordingly in automated fashion, or a notification for such a measure may be provided. Accordingly, the occurrence of dark spots may preferably represent an indication for a lack of cavitation and may be accordingly accompanied by an increase in the power of the laser beam or a corresponding notification.

Preferably, the determination of a parameter characterizing the effect on the eye by the apparatus comprises a determination of a structure and/or a texture of a part of the eye impinged by the laser beam and in particular the determination of a homogeneity of the structure and/or the texture of the part of the eye impinged by the laser beam. By way of example, a homogeneity or texture that deviates from a predetermined homogeneity or texture, in particular that is reduced in relation thereto, may be an indication for irregularity in the functionality of the apparatus. Preferably, this may be accompanied by the provision of the notification to the user and/or to the manufacturer of the apparatus and/or to a maintenance device.

Preferably, the determination of a parameter characterizing an external influence on the preparation and/or the implementation of the method for refractive surgery from outside of the apparatus comprises a determination of a movement path of at least one part of the apparatus during the preparation and/or the implementation of the method for refractive surgery, in particular along the optical axis of the eye. By way of example, the movement path of the contact glass and/or of an element carrying the contact glass can be determination in the process, preferably along the optical axis of the eye or perpendicular to the surface of the eye. Particularly preferably, the movement path of the part of the apparatus can be caused by a patient whose eye is intended to be treated exerting a force on the part of the apparatus and thereby causing a movement of the part of the apparatus. By way of example, such an exertion of force and/or such a bringing about of the movement path of the part of the apparatus may occur during the docking of the contact glass or of the apparatus on the eye and/or in an already docked state. Provided a movement path of the part of the apparatus is determined, this can for example indicate a movement of the patient which in turn can be an indication for, in particular, a restless emotional state of the patient. Such a recognition can preferably make an interruption and/or adaptation of the method appear advantageous and/or the output of a corresponding notification to the user of the apparatus appear advantageous in order to avoid a defective treatment on account of an unwanted relative movement of the eye relative to the apparatus.

Preferably, the determination of a parameter characterizing an external influence on the preparation and/or the implementation of the method for refractive surgery from outside of the apparatus comprises a determination of acoustic background noises before and/or during the preparation and/or the implementation of the method for refractive surgery. The occurrence of background noises may represent, for example, an indication for a restless emotional state of the patient and/or an indication for otherwise unsuitable conditions for implementing a refractive surgical method. By way of example, the occurrence or presence of background noises may be an indication for an increased risk of arising mechanical influences, for instance vibrations and/or tremors and/or relative movements between the eye and the apparatus. Thus, it may be advantageous, for example, to adapt and/or adjust the treatment or the method if background noises that significantly deviate from a predetermined intended value have been determined. Alternatively or in addition, it may be advantageous to output an appropriate notification, for example to the user of the apparatus, to draw attention to the background noises.

Preferably, the determination of a parameter characterizing a suitability of the effect of the eye by the laser beam for the further method of the refractive surgery on the eye comprises a determination of a time required for the surgical removal of a lenticule following the effect on the eye by the laser beam. Provided the apparatus is designed to remove the lenticule independently and/or in an automated manner, the time required to this end can preferably be determined in automated fashion and/or system-internally. Provided the removal of the lenticule is carried out differently, that is to say not by the apparatus, for instance manually by a surgeon and/or a user of the apparatus, the time period required for the removal of the lenticule can be determined, for example, by virtue of the apparatus requesting the surgeon and/or the user to specify the required time period within the scope of the query. By way of example, a corresponding query can be displayed on a screen. A specification of the time period required may be based on a measurement and/or an estimate, for example. Preferably, further information in relation to the removal of the lenticule and/or other activities to be implemented in this context and/or other circumstances can be queried, for example the difficulty and/or complexity of the removal of the lenticule. Preferably, a quality of the separation or of the detachment of the lenticule by the laser beam is deduced on the basis of information obtained in respect of the time period required for the removal of the lenticule. Thus, for example, an increase in the time period required for the removal of the lenticule may indicate a detachment of the lenticule by the laser beam that is worthy of improvement. By way of example, such a lengthening can be determined in the individual case, that is to say for each individual implementation of the method, in comparison with a predetermined intended value and/or with one or more values determined previously. Alternatively or in addition, there can be for example a statistical analysis of the time periods required for a multiplicity of implemented methods, said analysis preferably facilitating a correlation with other parameters of the method and/or of the apparatus. In this way, it is possible to recognize, for example, necessary maintenance for the system and a notification for such necessary maintenance can be provided accordingly, for example for the user and/or the manufacturer and/or a maintenance device. Alternatively or in addition, a notification can be provided, for example, in respect of necessary or advantageous schooling of the user and/or the surgeon.

Further details and advantages of the invention will now be explained more specifically on the basis of an exemplary embodiment illustrated in the following drawing. It is understood that the features and embodiments mentioned above and below should not only be considered part of the disclosure in the respective combination; instead, it is understood that other combinations of features that appear technically advantageous to a person skilled in the art and/or the features on their own should also be considered part of the disclosure relating to the monitoring of the functionality of an apparatus.

In detail:

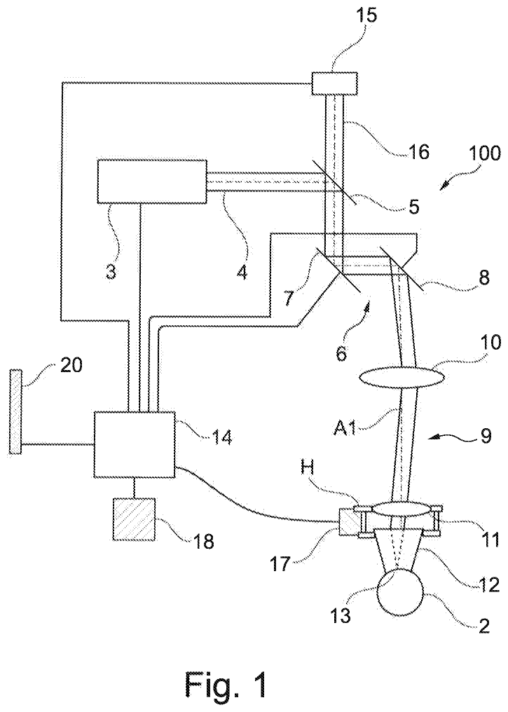

FIG. 1 shows, in a schematic illustration, an apparatus 100 according to a preferred embodiment of the invention for refractive surgery on an eye 2.

FIG. 1 shows an apparatus 100 according to a preferred embodiment of the invention for refractive surgery on an eye. The apparatus 100 is designed as a treatment appliance and serves to carry out, using a laser beam a refractive error correction on an eye 2 of a patient by means of a method for refractive surgery. To this end, the apparatus 100 comprises a laser or laser source 3, which emits pulsed laser radiation. In this case, the pulse duration is in the femtosecond range, for example, and the laser radiation acts on the cornea of the eye 2 in order to separate a lenticule from the surrounding cornea within the cornea.

The laser beam emitted by the laser 3 along an optical axis A1 or the treatment beam 4 is incident on a beam splitter 5 in the process, the latter guiding the laser beam 4 to a scanning device 6. The scanning device 6 has two scanning mirrors 7 and 8 which are rotatable about mutually orthogonal axes such that the scanning device 6 deflects the treatment beam 4 in two dimensions. An adjustable projection optical unit 9 focuses the treatment beam 4 onto or into the eye 2, and hence moreover complements the scanning device 6 such that three-dimensional scanning of the focused laser beam is facilitated. In this case, the projection optical unit 9 has two lens elements 10 and 11.

A contact glass 2 is disposed downstream of the lens element 11 and is connected securely to the lens element 11 by way of a holder H and hence to the beam path of the apparatus 100. The contact glass 12 rests on the cornea of the eye 2. The optical combination of contact glass 2 and the other optical components of the apparatus 100 causes the treatment beam 4 to be focused at a focus 13 located within the cornea of the eye 2.

The apparatus 100 further comprises a control unit 14 which is configured, in particular, to control the scanning device 6, the laser 3 and the projection optical unit 9. Moreover, the control unit 14 is configured to monitor the functionality of the apparatus and, for this purpose, determine at least one parameter of a preparation and/or implementation of a method for refractive surgery and analyze the functionality of the apparatus on the basis of the determined parameter. Even though the embodiment shown illustrates only one control unit, a plurality of control units which carry out the specified tasks and/or other tasks may also be provided in accordance with other embodiments.

The control unit 14 preferably determines the relative position of the focus 13, both transversely to the optical axis A1 (by the scanning mirrors 7 and 8) and in the direction of the optical axis A1 (by the projection optical unit 9).

Further, the control unit 14 reads a detector 15 which detects radiation scattered back and/or reflected by the cornea, which radiation passes through the contact glass and passes the beam splitter 5 as reflection radiation 16. To this end, there can be confocal imaging of the back-scattered reflection radiation 16 on the detector 15. Therefore, the detector 15 can represent a sensor, in particular one of the plurality of sensors which are connected to the control unit 14 for the purposes of monitoring the function of the apparatus.

For the treatment, the contact glass 12 is coupled to the eye 2 such that the contact glass 12 preferably rests directly on the eye. According to an embodiment, a liquid can additionally be arranged between the contact glass 12 and the eye 2 in order to obtain a suitable transition of the refractive indices between the contact glass 12 and the eye 2 and thereby minimize reflections and/or other losses of the laser beam and/or the reflection radiation 16. According to another embodiment, the contact glass 12, preferably a curved contact glass 12 adapted to the curvature of the cornea of the eye 2, rests directly on the cornea of the eye 2.

On account of the contact of the cornea with the contact glass 12, the eye 2 is in a predetermined relative position with respect to the contact glass 12 and hence with respect to the apparatus 100. As a result, the focus 13 can be positioned exactly in three dimensions in the cornea, but also within the contact glass 12, by way of an actuation of the scanning device 6 and the adjustable projection optical unit 9.

Moreover, according to the embodiment shown, the control unit 14 is connected to a motion sensor 17 and an acoustic sensor 18. According to the embodiment shown, the motion sensor 18 is arranged on the holder H connecting the lens element 11 to the contact glass 12 and accordingly follows a movement path of the contact glass, in particular along the optical axis A1 or the optical axis of the eye 2. In this way, the control unit 14 can determine the movement path of the contact glass as a parameter by the motion sensor 17 and can use this for the analysis of the functionality of the apparatus.

By means of the acoustic sensor 18, the control unit can further determine acoustic background noises as a parameter and use these for the analysis of the functionality of the apparatus 100. By way of example, the acoustic sensor 18 can be arranged in such a way that it can receive acoustic signals or reports especially from where the occurrence thereof is to be expected. By way of example, the acoustic sensor 18 can be directed at the head of the patient in order to be able to reliably recognize background noises caused by the patient. By way of example, such a parameter can characterize the one or more external influences from outside of the apparatus on the preparation and/or implementation of the method for refractive surgery. The acoustic sensor 18 can also represent a sensor which is connected to the control unit 14 for the purposes of determining parameters for monitoring the functionality of the apparatus.

Moreover, the control unit 14 as per the embodiment shown is connected to a display unit 20, for instance a computer display. The apparatus 100 is configured to output and/or record notifications via the display unit 20, for example for the user of the apparatus 100, and/or to query the user of the apparatus 100. Thus, for example a time period required for removing the lenticule from the eye 2 by the user or a surgeon can be determined as a further parameter by virtue of placing via the display unit 20 a corresponding query for the user or the surgeon, and requesting the latter to input a corresponding value. To the extent that there is an input, this determined time period can also be used as a parameter for the analysis of the functionality of the apparatus 100. By way of example, such a parameter can characterize the suitability of the effect on the eye 2 by the laser beam for the further method of the refractive surgery on the eye.

Additionally, the apparatus 100 or the control unit 14 can use the reflection radiation 16 to determine a parameter. By way of example, the control unit can make use of the detector 15 to this end. A parameter determined in this way can for example characterize the provision of the laser beam by the apparatus. Preferably, the determination of the parameter on the basis of the reflection radiation 16 may comprise the determination of possible damage to and/or contamination of the contact glass 12 and/or other optical components of the apparatus which influence the functionality of the apparatus 100. Moreover, in this way it is possible for example to determine a parameter which characterizes the effect on the eye 2 by the apparatus 100. By way of example, this may comprise the determination of the occurrence or lack of scattering centers at the positions at which the laser beam 4 acts on the eye 2, which may be implemented for example by determining bright and/or dark spots in the image of the reflection radiation 16.

On the basis of the determined parameters there can subsequently be an analysis of the functionality of the apparatus 100. The analysis of the functionality can preferably be carried out by the control unit 14 and/or another component of the apparatus 100. Alternatively or in addition, the parameters can preferably be provided to an external apparatus, for example a server, which thereupon implements the analysis and/or monitoring of the functionality of the apparatus.

Particularly preferably, monitoring of the apparatus is carried out over a multiplicity of implemented methods and comprises the collection of statistical data in respect of the parameters determined. In this way, it is preferably possible to also determine long-term changes which could not be determined if only individual methods or treatments were considered. In this way, there can be reliable monitoring of the functionality of the apparatus.

### LIST OF REFERENCE SIGNS

2 Eye
3 Laser
4 Laser beam
5 Beam splitter
6 Scanning device
7 Scanning mirror
8 Scanning mirror
9 Projection optical unit
10 Lens element
11 Lens element
12 Contact glass
13 Focus
14 Control unit
15 Detector
16 Reflection radiation
17 Motion sensor
18 Acoustic sensor
20 Display unit
100 Apparatus for refractive surgery on an eye
A1 Optical axis

1-15. (canceled)

16. A method for controlling a functionality of an apparatus for refractive surgery on an eye using a laser beam, the method comprising:
determining at least one parameter of a preparation and/or implementation of a refractive surgery, wherein the preparation and/or the implementation of the refractive surgery are carried out at least in part by the apparatus for refractive surgery using the laser beam; and
analyzing the functionality of the apparatus on the basis of the determined parameter of the preparation and/or the implementation of the refractive surgery,
wherein the at least one parameter characterizes one or more of the following properties of the preparation and/or implementation of the refractive surgery:
a provision of the laser beam by the apparatus;
an effect on the eye by the apparatus;
an external influence on the preparation and/or the implementation of the refractive surgery from outside of the apparatus; and
a suitability of the effect on the eye by the laser beam for a further proceeding of the refractive surgery on the eye.

17. The method as claimed in claim 16, further comprising:
receiving data from a sensor, wherein determining the at least one parameter of the preparation and/or implementation of the refractive surgery is based on the data from the sensor.

18. The method as claimed in claim 16, further comprising one or more of:
implementing a measure for improving the functionality of the apparatus;
providing a notification about the functionality of the apparatus; and
adjusting or modifying an operation of the apparatus.

19. The method as claimed in claim 16, wherein the analysis of the functionality of the apparatus on the basis of the determined parameter comprises:
comparing the determined parameter of the preparation and/or the implementation of the refractive surgery on the basis of the determined parameter with a predetermined intended value; and
assessing the functionality of the apparatus for refractive surgery on the basis of the comparison of the determined parameter with the predetermined intended value.

20. The method as claimed in claim 16, wherein the determination of a parameter characterizing the provision of the laser beam by the apparatus comprises a determination of a state of a contact glass of the apparatus, wherein the laser beam is applied to the eye through the contact glass.

21. The method as claimed in claim 20, wherein the determination of the state of the contact glass comprises a determination of optical structures in an image of optical radiation passing through the contact glass.

22. The method as claimed in claim 21, wherein the determination of the optical structures comprises a determination of optical edges in the image, wherein the determination of the optical edges comprises a determination of edges running in a straight line and/or edges running in a circle.

23. The method as claimed in claim 16, wherein the determination of the parameter characterizing the effect on the eye by the apparatus for the purposes of monitoring the functionality of the apparatus comprises a determination of a docking time of the apparatus on the eye and/or a determination of a number of necessary docking attempts until the apparatus successfully docks on the eye.

24. The method as claimed in claim 16, wherein the determination of the parameter characterizing the effect on the eye by the apparatus comprises a determination of arising optical structures in an image of the eye during application of the laser beam to the eye, wherein the arising optical structures comprise bright spots and/or dark spots.

25. The method as claimed in claim 16, wherein the determination of the parameter characterizing the effect on the eye by the apparatus comprises a determination of a structure and/or a texture of a part of the eye impinged by the laser beam and, optionally, a determination of a homogeneity of the structure and/or the texture of the part of the eye impinged by the laser beam.

26. The method as claimed in claim 16, wherein the determination of the parameter characterizing the external influence on the preparation and/or the implementation of the refractive surgery from outside of the apparatus comprises a determination of a movement path of at least one part of the apparatus during the preparation and/or the implementation of the refractive surgery along the optical axis of the eye.

27. The method as claimed in claim 16, wherein the determination of the parameter characterizing the external influence on the preparation and/or the implementation of the refractive surgery from outside of the apparatus comprises a determination of acoustic background noises before and/or during the preparation and/or the implementation of the refractive surgery.

28. The method as claimed in claim 16, wherein the determination of the parameter characterizing the suitability of the effect of the eye by the laser beam for the further proceeding of the refractive surgery on the eye comprises a determination of a time required for a surgical removal of a lenticule following the effect on the eye by the laser beam.

29. An apparatus for refractive eye surgery using a laser beam comprising a control unit, wherein the control unit is programmed to:
monitor the functionality of the apparatus by:
determining at least one parameter of a preparation and/or implementation of a refractive surgery, wherein the preparation and/or the implementation of the refractive surgery are carried out at least in part by the apparatus for refractive surgery using the laser beam; and
analyzing the functionality of the apparatus on the basis of the determined parameter of the preparation and/or the implementation of the for refractive surgery;
wherein the at least one parameter characterizes one or more of the following properties of the preparation and/or implementation of the refractive surgery:
a provision of the laser beam by the apparatus;
an effect on the eye by the apparatus;
an external influence on the preparation and/or the implementation of the refractive surgery from outside of the apparatus; and
a suitability of the effect on the eye by the laser beam for the further proceeding of the refractive surgery on the eye.

30. The apparatus as claimed in claim 29, further comprising a sensor, wherein monitoring the functionality of the apparatus further comprises:
receiving data from the sensor, wherein determining the at least one parameter of the preparation and/or implementation of the refractive surgery is based on the data from the sensor.

31. The apparatus of claim 29, wherein the control unit is further programmed to:
implement a measure for improving the functionality of the apparatus;
provide a notification about the functionality of the apparatus; and/or
adjust or modify an operation of the apparatus.

32. The apparatus of claim 29, wherein the analysis of the functionality of the apparatus on the basis of the determined parameter comprises:
comparing the determined parameter of the preparation and/or the implementation of the refractive surgery on the basis of the determined parameter with a predetermined intended value; and
assessing the functionality of the apparatus for refractive surgery on the basis of the comparison of the determined parameter with the predetermined intended value.

33. A method for refractive eye surgery using a laser beam of an apparatus for refractive surgery on the eye, the method comprising:
treating the eye by carrying out a refractive eye surgery with the apparatus while monitoring a functionality of the apparatus, wherein monitoring the functionality of the apparatus includes:
determining at least one parameter of a preparation and/or implementation of the refractive surgery, wherein the preparation and/or the implementation of the refractive surgery are carried out at least in part using the laser beam of the apparatus; and
analyzing the functionality of the apparatus based on the determined parameter of the preparation and/or the implementation of the refractive surgery,
wherein the at least one parameter characterizes one or more of the following properties of the preparation and/or implementation of the refractive surgery:
a provision of the laser beam by the apparatus;
an effect on the eye by the apparatus;
an external influence on the preparation and/or the implementation of the refractive surgery from outside of the apparatus; and
a suitability of the effect on the eye by the laser beam for the further proceeding of the refractive surgery on the eye.

34. The method as claimed in claim 33, further comprising one or more of:
implementing a measure for improving the functionality of the apparatus;
providing a notification about the functionality of the apparatus; and
adjusting or modifying an operation of the apparatus.

35. The method as claimed in claim 33, wherein the analysis of the functionality of the apparatus based on the determined parameter comprises:
comparing the determined parameter of the preparation and/or the implementation of the refractive surgery on the basis of the determined parameter with a predetermined intended value; and
assessing the functionality of the apparatus for refractive surgery on the basis of the comparison of the determined parameter with the predetermined intended value.
